(19) United States
(12) Patent Application Publication
(10) Pub. No.: US 2012/0157766 A1
(43) Pub. Date: Jun. 21, 2012

(54) DEVICE FOR ENDOSCOPIC TREATMENT OF UPPER GASTROINTESTINAL BLEEDING

(75) Inventors: Taarif Jafferi, Bangkok, MI (US); Raghunath Sai Katragadda, Ann Arbor, MI (US); Rahul Rattan, Ann Arbor, MI (US); Zachary T. Weingarden, West Bloomfield, MI (US); Robert L. Stoler, Ypsilanti, MI (US)

(73) Assignee: THE REGENTS OF THE UNIVERSITY OF MICHIGAN, Ann Arbor, MI (US)

(21) Appl. No.: 13/379,900

(22) PCT Filed: May 20, 2010
(86) PCT No.: PCT/US10/35539

§ 371 Date: Dec. 21, 2011

Related U.S. Application Data
(60) Provisional application No. 61/180,204, filed on May 21, 2009.

(51) Int. Cl. A61B 17/94

(52) U.S. Cl. 600/106

(57) ABSTRACT

A medical device designed for gastrointestinal endoscopists who require a more effective means of removing blood clots from the upper GI tract during episodes of upper GI bleeding. It is an endoscope attachment that breaks down blood clots, allowing them to be removed through the existing endoscope suction channel. Current methods are time-consuming, ineffective, and often life-threatening. This device offers an efficient alternative that makes the procedure quicker, safer, easier, and more cost-effective. The product offers simple solution to a life-threatening problem.

### CROSS-REFERENCE TO RELATED APPLICATIONS

This application claims the benefit of U.S. Provisional Application No. 61/180,204, filed on May 21, 2009. The entire disclosure of the above application is incorporated herein by reference.

### FIELD

The present disclosure relates to endoscopic treatment and, more particularly, relates to a device for endoscopic treatment of upper gastrointestinal bleeding.

### BACKGROUND AND SUMMARY

This section provides background information related to the present disclosure which is not necessarily prior art. This section also provides a general summary of the disclosure, and is not a comprehensive disclosure of its full scope or all of its features.

Upper gastrointestinal (GI) bleeding, which occurs most commonly in the stomach or duodenum, can be a very serious and life-threatening condition. Up to 10% of the yearly 300,000 hospitalizations in the US for acute upper GI bleeding result in patient mortality. A critical component of effective treatment is the physician's ability to quickly assess and treat the bleeding site.

Upon diagnosis of upper GI bleeding, the physician inserts an endoscope down the patient's esophagus and into the stomach and duodenum for visual assessment of the bleeding location. Endoscopes consist of an objective lens to allow vision and one or two instrument channels that allow various tools, water jets, or suction power to be delivered to the distal end of the endoscope inside the patient. If the bleeding site can be located, the physician can immediately treat the ulcer or alternative bleeding source. This is usually accomplished with the use of thermal probes such as a bipolar probe, clips or a sclerotherapy needle catheter used to inject diluted epinephrine through the instrument channel.

During upper GI bleeds, the patient's stomach is rarely clean and empty; rather, it is often filled with large coagulations of clotting blood. These blood clots are a great frustration to physicians as they attempt to locate the bleeding source, as they can rapidly fill the stomach and obstruct the objective lens. A common method to remove blood clots is with suction power through the endoscope instrument channel. However, these clots are often quite large and easily clog this narrow channel. This then requires multiple extractions and reinsertions of the endoscope, which is not only uncomfortable for the patient but time-consuming when time is critical. Sometimes, the physician will use endoscopic instruments such as snares or forceps to break the clots into smaller pieces, but this is also time-consuming and usually ineffective. Moreover, physicians are generally reluctant to use such techniques because of the risk of induced bleeding. Typically, patients are admitted to the intensive care unit (ICU) of the hospital and digestion is stimulated using a prokinetic drug such as erythromycin. After the stomach clears naturally, a second endoscopy is performed in attempt to locate the bleeding ulcer.

Extended patient stay, the use of expensive drugs, blood transfusions, and multiple endoscopies all add tremendous hospital costs. Additionally, the inability to quickly locate and address a bleeding ulcer can increase the patient's risk of death. In fact, if re-bleeding occurs, the chance of morality increases to 25%.

The Endocutter 10, according to the principles of the present teachings, is a disposable attachment that can be easily secured to the distal end of an endoscope, such as a single or dual channel endoscope, and is designed to break down blood clots during episodes of upper GI bleeding. A micro-motor at the distal end is powered through thin wires that run through the endoscope instrument channel. This motor spins a small blade at sufficient torque and speed to break down large blood clots and other stomach contents that would otherwise clog the endoscope. The suction power can be delivered in concurrence with the device, allowing for rapid and effective clearing of the stomach. The cutting blade is recessed within a clear plastic protective casing. This prevents accidental contact with the stomach lining while still preserving adequate vision through the objective lens of the endoscope.

If the initial endoscope examination, with a conventional endoscope, reveals cumbersome blood clots in the stomach, the physician would retract the endoscope, pass wiring through the instrument channel, and attach the Endocutter 10 of the present teachings, which is compatible with any endoscope. As suction power draws blood clots toward the channel, the physician breaks them down by powering the motor with a simple on/off switch. As blood clots are removed, the physician can search for the bleeding site. If the bleeding is severe and the physician is reluctant to break visual contact, the Endocutter 10 is designed to accommodate the use of a therapeutic device such as a sclerotherapy needle catheter, bipolar gold probe, or endoscopic clip device through the endoscope's second instrument channel, if a double-channel scope is in use.

The Endocutter 10 allows quick removal of obstructive blood clots and stomach contents from the patient's stomach during upper GI bleeding. This allows for immediate treatment of bleeding ulcers or alternative bleeding sources, for e.g., arteriovenous malformations, varices, mucosal tears, greatly increasing the effectiveness of the procedure. This can significantly decrease hospital procedure costs, reduce physician fatigue and frustration, and most importantly, save patient lives.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of a device for endoscopic treatment of upper gastrointestinal bleeding according to the principles of the present teachings;

FIG. 2 is a top view of the device of FIG. 1;

FIG. 3 is a front view of the device of FIG. 1;

FIG. 4 is a side view of the device of FIG. 1;

FIG. 5 is a schematic view of a motor used in the device of FIG. 1; and

FIG. 6 is an endoscope having the device of FIG. 1 coupled thereto.

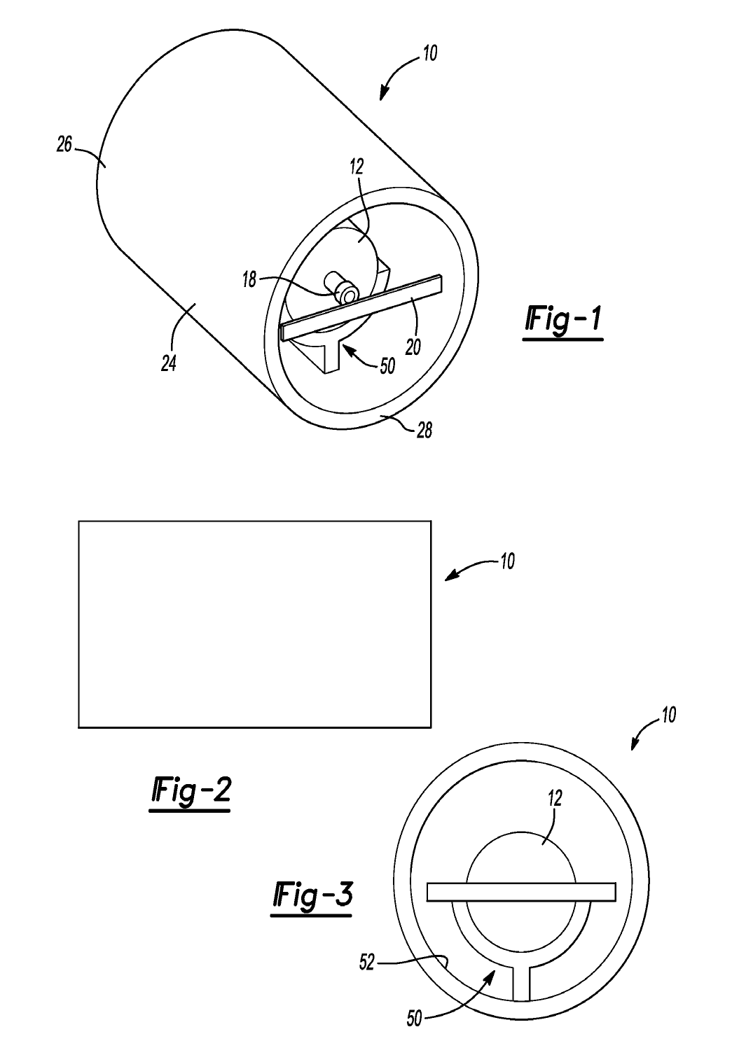

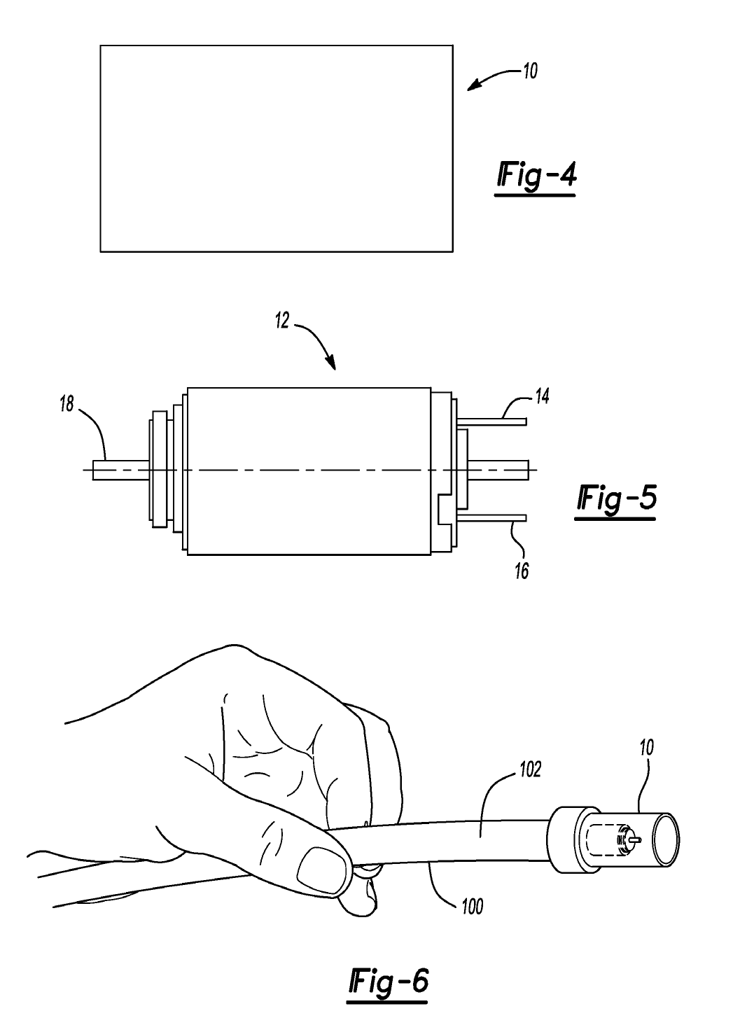

Corresponding reference numerals indicate corresponding parts throughout the several views of the drawings.

### DETAILED DESCRIPTION

Example embodiments will now be described more fully with reference to the accompanying drawings. Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

According to the principles of the present teachings, a device for endoscopic treatment of upper gastrointestinal bleeding (generally referred to as Endocutter 10) is provided having advantageous construction and method of use. The Endocutter 10 of the present teachings can employ mechanical power to break blood clots and other stomach contents down into pieces that are small enough to pass through the conventional endoscope vacuum channel. The Endocutter 10, which can be in the form of a releasable attachment, is designed to prevent damage to the stomach mucosa by the spinning blade while still preserving the functions of an endoscope, and is used in conjunction with a method that efficiently removes clots and improves the overall effectiveness of the procedure.

Referring now to the figures, Endocutter 10 is an endoscope accessory that is designed to be compatible with any single or dual channel endoscope 100. The device 10 is used in conjunction with the existing suction power of endoscope 100 and is operable to chop or otherwise cut blood clots and other stomach content into smaller pieces before such pieces enter the narrow instrument channel of the conventional endoscope. Generally, Endocutter 10 comprises a motor 12 having a pair of electrical input contacts 14, 16, an internal drive system for converting electrical power received through the pair of electrical input contacts 14, 16 to rotational drive power via an output shaft 18. Motor 12 rotatably supports a spinning cutting blade 20 operably coupled to output shaft 18 of motor 12 for rotation therewith. Cutting blade 20 can comprise, in some embodiments, one or more sharpened edge (or beveled) regions 22 to aid in cutting. However, it should be appreciated that non-sharpened edges can also be used. Still further, in some embodiments, cutting blade 20 can be shaped to promote a desired circulating flow of material, such as by way of a pitched or otherwise inclined cutting blade or propeller.

According to the present teachings, cutting blade 20 is encased within a clear polycarbonate casing 24. In some embodiments, casing 24 is a cylindrical member being made of polycarbonate, such as a transparent biocompatible plastic material, having a proximal end 26 and a distal end 28. It should be appreciated that casing 24 can have alternative shapes, including a venturi shape, a converging or diverging cone shape, or any other shape conducive for use within a gastrointestinal system and in conjunction with material flow. In some embodiments, cutting blade 20 is generally surrounded and recessed within casing 24 in order to protect the stomach and upper GI tract lining from contact with cutting blade 20. In this way, cutting blade 20 is inwardly spaced from distal end 28 of casing 24 a sufficient distance to prevent or at least minimize the occurrence of stomach lining intruding within an inner volume of casing 24 and contacting cutting blade 20. In some embodiments, motor 12 is sized sufficiently small to be placed within casing 24, while preserving vision through the objective lens and use of the instrument channel for the flow of material.

In some embodiments, proximal end 26 of casing 24 can be attached or otherwise fastened to a distal end 102 of endoscope 100. In some embodiments, this can be achieved using a rubber attachment member generally surrounding Endocutter 10 and endoscope 100. The rubber attachment member can be sized to exert a reliable and simple connection between Endocutter 10 and endoscope 100. In this way, power lines (not shown) that are electrically coupled to electrical input contacts 14, 16 can extend through an internal channel of conventional endoscope 100 to provide electrical energy to motor 12. The power lines can be coupled to a power source, such as a DC power source. A switch (not shown) can be used to actuate motor 12. Endocutter 10 thus permits the physician to quickly locate the bleeding site without the need for repeated endoscope reinsertions or extended patient stay.

In some embodiments, by way of non-limiting example, Endocutter 10 can be 30 mm long with an outer diameter of 14.6 mm (similar in diameter to dual-channel endoscopes). The hydrophobic-coated polycarbonate casing 24 can be 25 mm long, 1 mm thick, and transparent to maximize the field of vision. A motor holder 50 can extend from an inner wall 52 of casing 24 to support motor 12 and can define an inner diameter of 5.5 mm and a curvature angle from 45 degrees to 360 degrees (or any curvature in between) to receive and secure the motor 12. Motor holder 50 can extend the length of motor 12, such as for example about 16 mm. Motor holder 50 can comprise a stem 54 connecting motor holder 50 to inner wall 52 of casing 24.

Generally, in some embodiments, motor 12 is 6 mm in diameter, 22.9 mm in length, and the tip of the motor shaft is placed 1 mm from distal end 28 of casing 24. The cutting blade 20 is connected to the output shaft 18 using a blade connector placed over the shaft. The cutting blade 20 can, in some embodiments, be 10 mm in length, 1 mm in width, and 0.1 mm in thickness and can be passed over the spindle of the blade connector and glued using industrial stainless steel adhesive. The terminals of the motor can be enclosed in a wire cap to ensure robust fastening of wires. A rubber attachment can be fixed over the other end of the casing and secures the Endocutter 10 at the distal end 102 of the endoscope 100.

It should be appreciated that the motor placement and blade size are specifically designed to maximize cutting ability while still maintaining use of the endoscope's resources (i.e. the objective lens and instrument channels). The device is also compatible with current therapeutic measures that are required during upper GI bleeding, so the physician does not have to retract and reinsert the endoscope for active-bleed treatment. It should also be appreciated that case size and motor size are specifically designed to permit unobstructed flow of cut blood clots and other stomach content passed the motor, within the internal volume of the casing, and subsequently into the vacuum lines of the endoscope. It has been found that in some embodiments, 80% or less obstruction can suffice; however, in some embodiments, 50% or less obstruction provides improved performance. Such measurements can be obtained by defining a cross-sectional area along a plane orthogonal to a longitudinal axis of the motor and/or casing and comparing the same to an internal cross-sectional area of the casing along the same plane.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A medical device for attachment to an endoscope, said medical device comprising:
a casing connectable with the endoscope, said casing defining an internal volume;
a motor at least partially disposed within said internal volume, said motor having a rotatable output shaft; and
a blade member operably coupled with said rotatable output shaft for rotation therewith to produce a cutting motion.

2. The medical device according to claim 1, wherein said motor and said casing are sized such that a volume of said motor is less than said internal volume resulting in a flow-by volume, said flow-by volume being sufficient sized to permit blood clots or other stomach content to flow therethrough.

3. The medical device according to claim 1, wherein said motor defines a cross-sectional area along a plane orthogonal to a longitudinal axis thereof that is less than 80 percent of an internal cross-sectional area of said casing along said plane.

4. The medical device according to claim 1, wherein said motor defines a cross-sectional area along a plane orthogonal to a longitudinal axis thereof that is less than 50 percent of an internal cross-sectional area of said casing along said plane.

5. The medical device according to claim 1 wherein said casing is cylindrical.

6. The medical device according to claim 1, further comprising:
a motor support extending from an inner surface of said casing within said internal volume, said motor support supporting said motor.

7. The medical device according to claim 1 wherein said casing is transparent.

8. The medical device according to claim 1 wherein said blade member comprising a cutting edge.

9. The medical device according to claim 8 wherein said cutting edge is beveled.

10. The medical device according to claim 1 wherein said blade member is shaped to encourage flow through said internal volume.

11. A medical device for attachment to an endoscope, said medical device comprising:
a cylindrical casing connectable with the endoscope, said casing defining an internal volume having a motor support extending within said internal volume;
a motor at least partially disposed within said internal volume and supported by said motor support, said motor having a rotatable output shaft; and
a blade member operably coupled with said rotatable output shaft for rotation therewith to produce a cutting motion,
wherein said motor defines a cross-sectional area along a plane orthogonal to a longitudinal axis thereof that is less than 80 percent of an internal cross-sectional area of said casing along said plane. said plane.

12. The medical device according to claim 11 wherein said casing is transparent.

13. The medical device according to claim 11 wherein said blade member comprising a cutting edge.

14. The medical device according to claim 13 wherein said cutting edge is beveled.

15. The medical device according to claim 11 wherein said blade member is shaped to encourage flow through said internal volume.
